(19) United States
(12) Patent Application Publication
(10) Pub. No.: US 2022/0313879 A1
(43) Pub. Date: Oct. 6, 2022

(54) CALCULATION DEVICE AND DIALYSIS APPARATUS

(71) Applicant: Fresenius Medical Care Deutschland GmbH, Bad Homburg (DE)

(72) Inventor: Andreas Maierhofer, Schweinfurt (DE)

(21) Appl. No.: 17/616,865

(22) PCT Filed: Jun. 3, 2020
(86) PCT No.: PCT/EP2020/065282

§ 371 Date: Dec. 6, 2021

(30) Foreign Application Priority Data
Jun. 7, 2019 (DE) 10 2019 115 553.3

(51) Int. Cl. A61M 1/16; A61M 1/34; G16H 20/40; G16H 40/63

(52) U.S. Cl. CPC A61M 1/1603; A61M 1/1611; A61M 1/3403; G16H 20/40; G16H 40/63; A61M 2205/502; A61M 2205/584; A61M 2205/3303

(57) ABSTRACT

The present disclosure relates to a calculation device for determining an interdialytic sodium intake of a patient and/or for determining a non-osmotically triggered interdialytic liquid intake, including a storage device and/or an input device configured for storing or for entering parameter values of the patient; a computing device, configured for calculating the interdialytic sodium intake of the patient and/or for calculating his non-osmotically triggered interdialytic liquid intake; and an output device for outputting a signal for controlling or closed-loop controlling a communication device and/or a medical blood treatment apparatus.

### CROSS-REFERENCE TO RELATED APPLICATIONS

The present application is the national stage entry of International Patent Application No. PCT/EP2020/065282, filed on Jun. 3, 2020, and claims priority to Application No. 10 2019 115 553.3, filed in the Federal Republic of Germany on Jun. 7, 2019, the disclosures of which are expressly incorporated herein in their entireties by reference thereto.

### TECHNICAL FIELD

The present disclosure relates to a calculation device as disclosed herein, to a medical blood treatment apparatus as disclosed herein, and to a method as disclosed herein.

### BACKGROUND

Numerous studies prove that in the common population, increased table salt intake leads to an increased occurrence of cardiovascular diseases, and that cardiovascular incidents like heart attack and stroke could be reduced by reducing the intake of table salt (NaCl). In particular, increased table salt intake leads, in part of the population, to increased blood pressure. Recent studies also suggest a negative impact on the immune system (Afar et al., Salt Intake and Immunity, Hypertension 72 p. 19ff; Evans et al., Emerging evidence of an effect of salt on innate and adaptive immunity, NDT, published online 2018, December 5).

People with impaired renal function are dependent on the withdrawal of excess sodium by dialysis. It is therefore particularly relevant for them to know the amount of salt they have taken in. Nutritional counseling and following the rules learned thereby may be helpful.

Nutrition counseling currently takes place mainly on the basis of nutrition tables. In terms of salt content, there are tables containing the typical salt content of food. In convenience food, there are also data relating to the total product, typical portions or per weight unit. At the same time, the actually consumed products and quantities must be determined for a calculation. Said determining proves regularly to be difficult in practice.

### SUMMARY

It is an advantage of the present disclosure to propose a calculation device for the sodium intake of dialysis patients and a further medical blood treatment apparatus.

The advantages are achieved by a calculation device as disclosed herein. The advantages are further achieved by the medical blood treatment apparatus as disclosed herein.

The calculation device described herein is configured for determining an interdialytic (i.e., between two consecutive dialysis sessions, in particular since the last performed or previous dialysis session (also in short: dialysis)) and/or daily sodium intake (herein also referred to as mᵢₙₜₑᵣ) of a patient (also: dialysis patient, although mostly referred to in short as patient). Alternatively or in addition thereto, the calculation device is configured for determining an interdialytic drinking amount, e.g., not triggered or induced osmotically interdialytic drinking amount or liquid intake (herein also referred to as V_{excess drink}).

For this purpose, the calculation device includes a storage device and/or an input device, further a computing device and an output device.

The storage device is configured for storing parameters (or parameter specifications or parameter values) of the patient. The input device is configured for entering parameter values of the patient.

The computing device is configured and/or programmed for calculating the interdialytic sodium intake mᵢₙₜₑᵣ of the dialysis patient and/or for calculating the interdialytic liquid intake V_{excess drink}.

The calculation device may be based on stored formulas or algorithms, such as disclosed herein. The calculation may be based alternatively or additionally on the parameter values retrievable from the storage device or from the input device by the calculation device.

The output device may be configured for outputting a signal for controlling a communication device and/or for controlling or closed-loop controlling a medical blood treatment apparatus.

The communication device may be designed as, or include, an output device, monitor, display, printer, database, etc.

The communication device may optionally be part of, or respectively connected to, the calculation device or the medical blood treatment apparatus.

A medical blood treatment apparatus (in short: treatment apparatus) is further described below.

The blood treatment apparatus includes:
fluid lines, which encompass at least one dialysis liquid inlet line and/or one dialysate outlet line, which are optionally connected to each other in fluid communication, for instance by a connector;
at least one conveying device for conveying a dialysis liquid within the dialysis liquid inlet line and/or within the dialysate outlet line; and
at least one control and/or closed-loop control device.

The medical blood treatment apparatus is configured to be connected to a dialysis liquid chamber of a blood filter respectively by the dialysis liquid inlet line and by the dialysate outlet line, which blood filter includes in addition to a dialysis liquid chamber a blood chamber, wherein dialysis liquid chamber and blood chamber are separated from each other by a semipermeable membrane.

The control device and/or closed-loop control device is configured for prompting or effecting a blood treatment using the medical blood treatment apparatus by hemofiltration, hemodialysis, or hemodiafiltration. The control device and/or closed-loop control device is connected in signal transmission to, or includes, a calculation device as disclosed herein.

In all of the following statements, the use of the expression “may be” or “may have” and so on, is to be understood synonymously with “preferably is” or “preferably has,” and so on respectively, and is intended to illustrate embodiments.

Whenever numerical words are mentioned herein, the person skilled in the art shall recognize or understand them as indications of numerical lower limits. Hence, unless it leads to a contradiction evident for the person skilled in the art, the person skilled in the art shall comprehend for example “one” as encompassing “at least one”. This understanding is also equally encompassed by the present invention as the interpretation that a numerical word, for example, “one” may alternatively mean “exactly one”, wherever this is evidently technically possible in the view of the person skilled in the art. Both of these understandings are encompassed by the present invention and apply herein to all used numerical words.

Whenever the terms “programmed” or “configured” are mentioned herein, it is thus disclosed that these terms are interchangeable.

Whenever a suitability or a method step is mentioned herein, the present disclosure encompasses a corresponding programming or configuring of a suitable apparatus or a section thereof—e.g., the blood treatment apparatus—as well as apparatuses programmed in such a manner.

Advantageous developments are subject-matter of the described embodiments.

Whenever an embodiment is mentioned herein, it is then an exemplary embodiment.

Embodiments may include one or several of the features mentioned supra and/or in the following in any combination which is technically possible.

When it is discussed herein that a value is calculated based on a parameter value (or other values), this may encompass that the calculation is based on an estimate of the parameter value or on an approximation of the parameter value (or of or to the other values).

In several embodiments, the computing device is configured and/or programmed (both terms are herein interchangeable) for calculating the interdialytic sodium intake mᵢₙₜₑᵣ and/or the interdialytic liquid intake V_{excess drink} based on the plasma sodium concentration cₚᵣₑ(n) prevalent at the beginning of the dialysis session and/or the sodium concentration in the urine cᵤᵣᵢₙₑ.

In several embodiments, the interdialytic sodium intake mᵢₙₜₑᵣ and/or the interdialytic liquid intake V_{excess drink} are calculated using one of the formulas 1, 3, 5, 8, 11, 12, 13, 14, 17 mentioned herein.

Whenever the interdialytic liquid intake or the liquid intake V_{excess drink} is mentioned herein, understood thereunder may be a liquid volume to be calculated herein (and preferably to be removed by dialysis). It may be a liquid volume which the patient has taken without this being necessary for maintaining the fluid balance. Therefore, it is optionally referred to herein as the liquid intake V_{excess drink}.

In some embodiments, the computing device is directly or indirectly in signal communication with components of the medical blood treatment apparatus. The signal communication is thereby provided or configured such that values, as for example the plasma sodium or an interdialytic salt transfer, which values are measured by the medical blood treatment apparatus, during a dialysis session using the blood treatment apparatus, are transferred to the computing device, e.g., by the input device or by the communication device. This may be done by requesting these values from the medical blood treatment apparatus. It may, however, also be done by a sending function which is output by the medical blood treatment apparatus. Based on these values, calculating the interdialytic sodium intake and/or the interdialytic liquid intake may be carried out or repeated several times during the running dialysis session. Since later measuring values are usually more accurate than those collected at an early stage, this procedure may lead to more accurate calculation results for sodium intake and/or liquid intake.

In several embodiments, the output device, which is configured for outputting a signal for controlling a communication device, is further configured for displaying, outputting and/or storing values for the interdialytic sodium intake and/or for the interdialytic liquid intake on a display device as an example for a communication device which may be or may include a monitor, a display, a printer a storage element or a database or the like.

In some embodiments, the output device, which is configured for outputting the signal for controlling a communication device, is further configured for specifying a prescription based on the interdialytic sodium intake and/or on the non-osmotically triggered interdialytic liquid intake. The prescription may relate to the current or to a pending dialysis session. It may be an influence on the machine-adjustable treatment parameters.

In several embodiments, the output device is configured for controlling the medical blood treatment apparatus, by the signals sent by said output device to the medical blood treatment apparatus, such that the current dialysis session or the blood treatment ends when or once the determined interdialytic sodium intake and/or interdialytic liquid intake has been withdrawn from the treated blood.

In some embodiments, the calculation device or any of its components, devices, or apparatuses is configured to read values of at least one or more of the following parameters in any combination, wherein said reading may be done from, e.g., the input device and/or the storage device:
V distribution volume of the patient, i.e., the water content of the patient without overhydration or after removing his overhydration by dialysis. This parameter may be determined from, e.g., anthropometric formulas (e.g., the Watson formula) or by bioimpedance measurements;
ΔV interdialytic liquid excess; usually corresponds to the interdialytic weight gain. A liquid withdrawal V_{VF}=ΔV is often prescribed for the dialysis session;
cₚᵣₑ(n) plasma sodium concentration at the beginning of the current dialysis session (dialysis session n); it is determined from, e.g., laboratory measurements or from conductivity-based online determination by OCM (Online Clearance Monitoring) (cf. the EP 3 183 013 A1 or WO 2016/026569 A1 mentioned herein);
cₚₒₛₜ(n−1) plasma sodium concentration at the end of the previous dialysis session (referred to as dialysis session n−1); it may be, e.g., determined from laboratory measurements or from conductivity-based online determination, e.g., by OCM. This value is stored, e.g., at the end of the previous dialysis session n−1, as patient-related value on a suitable storage medium such as the storage device mentioned herein, and is made available again in the current dialysis session;
Vᵤᵣᵢₙₑ accumulated residual urine excretion between the contemplated dialysis session n and n−1. This value may be determined by the patient at home (keyword: urine collection). With constant habits, a representative value may be stored for the patient, advantageously as a daily amount (alternatively as a volume), so that the total amount may be calculated therefrom for differently long interdialytic intervals;
cᵤᵣᵢₙₑ sodium concentration in the urine, e.g., determined from laboratory measurements or as an assumption from plausibility considerations. For example, this value and its possible error may be estimated depending on Vᵤᵣᵢₙₑ: since there is a severe renal insufficiency in the dialysis patient, cₚᵣₑ(n) may be assumed as an upper limit for this value. As a lower limit, for example the dilution capacity of the kidney possible in this case may be assumed, e.g., c_{pre,min}=50 mmol/l;
N_{d} number of days between the end of the previous dialysis session n−1 and the current dialysis session n, determined by the current date and the stored date of the previous dialysis session.

When dialysis liquid is mentioned herein, it thus refers in doubt to the fresh fluid led to the dialysis liquid chamber of the blood filter via the dialysis liquid inlet line on the machine side. When dialysate is mentioned herein, it thus refers in doubt to the fluid removed from the dialysis liquid chamber of the blood filter via the dialysate outlet line on the machine side.

In several embodiments, the signals output to the communication device is transferred while also stating the qualitative and/or a quantitative accuracy of the signal or that of a value transmitted by the signal, the size of an error, an uncertainty, a possible value range (e.g., of a confidence interval) etc. of signal or value, for example of the interdialytic sodium intake mᵢₙₜₑᵣ and/or the interdialytic liquid intake V_{excess drink}, or relating thereto. Also possible is stating a standard deviation, variance, a color coding optionally with multiple colors, a traffic light display, etc. This may serve for a better understanding of an associated, displayed value.

Thus, in some embodiments, the qualitative and/or quantitative accuracy of the value may be communicated or will be communicated by numerically indicating an error (e.g., standard deviation) or a possible range of values (e.g., confidence interval) or by another reference to the reliability of the value, e.g., by color according to a traffic light model or sample.

In some embodiments, the qualitative and/or quantitative accuracy may be or may encompass the size of an error, an uncertainty and/or an imprecision.

In some embodiments, it may be configured that several or all parameter values are either entered directly to the medical blood treatment apparatus, or read by the dialysis device from external sources (storage medium, network, etc.). It is also possible that the calculation of, e.g., the interdialytic, daily and average daily salt intake and/or the liquid amount is carried out on external devices and the measured values of the blood treatment apparatus needed for the calculation are continuously transmitted to the external device.

A “dialysis session” may, for example, be a treatment unit by hemodialysis, hemofiltration, hemodiafiltration and/or a cell separation method and may be provided for the treatment and/or the purification of blood. For carrying out such blood treatment, a suitable blood treatment apparatus is used.

In hemodialysis, there is a concentration balance of small molecular substances between blood and a dialysis liquid over a semipermeable membrane, which separates the blood side and the hydraulic side of the blood treatment apparatus from each other. In this way, toxins and other kidney-related substances are withdrawn from the blood to be purified and received by the dialysis liquid.

In some embodiments, the blood treatment apparatus is prepared to vary the sodium content of the dialysis liquid controlled by the control device and/or the closed-loop control device.

Sensors may be provided for determining the temperature-compensated conductivity as well as the liquid flow upstream and downstream of the blood filter. These may be designed for determining the temperature-compensated conductivity, for ion-selective measurements, or for measurements according to other methods.

Based on obtained sensor values, in several embodiments, the control device or closed-loop control device carries out mathematical calculations for determining the electrolyte and liquid balance. Likewise, it can determine the default value for the electrolyte and liquid balance to be achieved in the current treatment based on user specifications and stored algorithms. User specifications and displaying the calculated quantities or the treatment progress are possible, for example via a user interface.

For a possible calculation of mᵢₙₜₑᵣ or of V_{excess drink}, considerations and formulas mentioned in the following or given elsewhere herein may be used exemplarily:

If a patient in the so-called single-pool model is described by a distribution volume V (e.g., normohydrated, i.e., without overhydration) with the sodium concentration c, thus, the following applies to the sodium balance between two dialysis sessions:

$${m}_{inter} = {V}_{pre} ( n ) {c}_{pre} ( n ) - {V}_{post} ( n - 1 ) {c}_{post} ( n - 1 ) + {\sum }_{j} {V}_{urine , f} {c}_{urine , f} \qquad Formula 1$$

In this, (n) means the state at the current dialysis session and (n−1) the state of the very previous dialysis session, the indices “pre” and “post” indicate the time, namely at the beginning or at the end of the dialysis session. The index “urine” designates values in the urine, wherein index “j” herein designates all urine collections in the interdialytic interval. If there is no residual diuresis left, then V_{urine,f}=0.

The distribution volumes Vₚᵣₑ and Vₚₒₛₜ may be determined from, e.g., bioimpedance measurements. However, it may be more practical and optionally more precise to determine only one of these values directly and to additionally, e.g., assume that the volume difference ΔV relative to V corresponds to the interdialytic weight gain, which may be determined by weighing the patient after and before the dialysis session:

$${V}_{pre} = {V}_{pre} ( n ) = {V}_{post} ( n - 1 ) + Δ V = V + Δ V \qquad Formula 2$$

Thus, the following applies:

$${m}_{inter} = V ( {c}_{pre} ( n ) - {c}_{post} ( n - 1 ) ) + Δ {VT}_{pre} ( n ) + {\sum }_{f} {V}_{urine , f} {c}_{urine , f} \qquad Formula 3$$

mᵢₙₜₑᵣₙ corresponds to the interdialytic sodium intake. By laboratory measurements of the blood-sodium concentration before and after the dialysis session and by collecting the urine between the considered dialysis sessions n and n−1 and laboratory measurements of the sodium concentration in the collected urine from interdialytic interval, the interdialytic salt intake of the patient may thus be determined without the need to analyze the nutrition or diet.

However, since frequent blood analyses are not practical in everyday clinical routine, an automated and/or calculated determination of the salt balance is extremely advantageous.

For this purpose, it is assumed that the interdialytic volume increase ΔV is completely compensated by the interdialytic liquid withdrawal V_{UE} using ultrafiltration in the subsequent treatment.

With the withdrawal of liquid, a sodium withdrawal m_{UX} takes place simultaneously:

$${m}_{UF} = {\int }_{{V}_{UF}} {c}_{do} dV \qquad Formula 4$$

In this, c_{do} represents the sodium concentration downstream of the dialyzer. This may be determined by the temperature-compensated conductivity measured in the effluent dialysate by a kinetic model for the influence of the concentration of electrolytes other than sodium, e.g., potassium (cₓ) and bicarbonate (cₘᵢₙ), (cf. EP 2 413 991 B1).

$${c}_{do} ( t ) = f ( CD ( t ) , {c}_{K} ( t ) , {c}_{Bic} ( t ) ) \qquad Formula 5$$

m_{UX} corresponds, in case of absence of renal residual excretion, to the interdialytic sodium intake when the plasma sodium concentration does not change by dialysis (i.e., by isonatremic dialysis), i.e., when no salt transfer between blood and dialysate takes place during the dialysis session. This is the case when blood sodium concentration and dialysate sodium concentration differ only slightly. In this case the following applies:

$${m}_{inter} ( {V}_{urine} = 0 ) = {m}_{UF} = {V}_{UF} {c}_{do} = {V}_{UF} {c}_{one} . \qquad Formula 6$$

Otherwise, the change in plasma sodium concentration may be taken into consideration by diffusive transfer m_{diff} between dialysate and blood during the dialysis session:

$${c}_{post} = {c}_{pre} = \frac{{m}_{diff}}{V} \qquad Formula 7$$

Therefore, this generally applies:

$${m}_{inter} ( {V}_{urine} = 0 ) = {m}_{UF} - {V}_{UF} ( {c}_{post} - {c}_{pre} ) = {m}_{UF} - {V}_{UF} \frac{{m}_{diff}}{V} \qquad Formula 8$$

The calculation of m_{diff} on the dialysate side by continuous measurement of the temperature-compensated conductivity upstream and downstream of the dialyzer or blood filter when corrected by a kinetic model is also disclosed in EP 2 413 991 B1.

If the predialytic plasma sodium concentration corresponds to a normal physiological state, then it is advantageous to adjust the dialysis liquid sodium concentration such that m_{diff}=0. This state is reached when, during the interdialytic interval, the salt intake is compensated by a corresponding drinking amount water so that the plasma sodium concentration does not change. If this is not the case, e.g., because the patient drinks more than needed for compensating the salt intake, for reasons not relating to his sensation of thirst, and thus takes in the liquid amount that is herein denoted as not osmotically triggered, the resulting plasma sodium concentration represents a pathological state. By diffusive salt transfer during the dialysis session, namely m_{diff}=0, this state may be compensated again. This can be described in the physiological concept of “free water removal”: In a functioning kidney, a positive “Free Water Clearance” means that the sodium concentration in the urine is lower than in the plasma, meaning that the kidney retains sodium and thus increases the plasma sodium concentration. On the other hand, with a negative “Free Water Clearance”, the sodium concentration in the urine is higher than that in the plasma, meaning that the kidney excretes sodium and thus decreases the plasma sodium concentration. This can be applied to the parameters relevant during the dialysis session as follows:

The “free water removal” V_{FWR} denotes thereby the (virtual) volume of salt-free water which is withdrawn from (V_{FWR}>0), or delivered to (V_{FWR}<0), the patient who has a distribution volume V, in order to change his plasma sodium concentration from cₚᵣₑ to cₚₒᵣₜ. This change corresponds to the change in plasma sodium concentration by the diffusive salt transfer during the dialysis session:

$${c}_{post} = \frac{( V + {V}_{UF} ) {c}_{pre}}{( V + {V}_{UF} ) - {V}_{FWR}} = {c}_{pre} + \frac{{m}_{diff}}{V} \qquad Formula 9$$

Algebraic transformation according to V_{FWR} results in:

$${V}_{FWR} = \frac{( V + {V}_{UF} ) {m}_{diff}}{{m}_{diff} + {Vc}_{pre}} \qquad Formula 10$$

Analogously, the drinking volume V_{excess drink} in the interdialytic interval (i.e., the time between two consecutive dialysis sessions n−1 and n) can be determined, which has led to a change of the plasma sodium concentration, and which the patient drunk, so to speak, “over the eight” in the interdialytic interval

$${V}_{excessdrink} = \frac{( {c}_{post} ( n - 1 ) - {c}_{pre} ( n ) ) ( V + {V}_{UF} )}{{c}_{post} ( n 1 )} \qquad Formula 11$$

This additional liquid intake has then led to a shift in his “physiological” plasma sodium value to a pathological condition.

By removing V_{FWR}=V_{excess drink}, e.g., by an increase in plasma sodium concentration by dialysis, this drinking volume is compensated again.

If a residual diuresis is present, the amount of salt absorbed through the diet is greater than the amount determined during the dialysis session. In order to determine the salt amount excreted via the urine, for example, the sodium concentration cᵤᵣᵢₙₑ is needed. This may be done, for example, by laboratory measurement in urine collection under typical conditions.

With decreasing residual excretion, the ability of the kidney to concentrate the sodium concentration is lost increasingly, so that the sodium concentration in the urine cᵤᵣᵢₙₑ increasingly approaches the plasma sodium concentration. Thus, an estimate may be made under these conditions:

$${m}_{inter} = {m}_{UF} - {V}_{UF} \frac{{m}_{diff}}{V} + {V}_{urinetot} {c}_{urine , avg} \\ ≅ {m}_{UF} - {V}_{UF} \frac{{m}_{diff}}{V} + {V}_{urinetot} {c}_{pre} \qquad Formula 12$$

For better understanding of daily eating and drinking habits, it is beneficial to refer V_{FWR} and mᵢₙₜₑᵣₙ to daily quantities by dividing these values by the number N_{d} of days in the interdialytic interval.

$${m}_{inter , d} = \frac{{m}_{inter}}{{N}_{d}} , {V}_{FWR , d} = \frac{{V}_{FWR}}{{N}_{d}} \qquad Formula 13$$

In the common dialysis regime of three dialysis sessions per week, the interdialytic interval over the weekend is one day longer than the other two intervals, so that in this interval the accumulation of liquid and salt in the patient is increased compared to the other two intervals. Since, however, the dialysis session takes the same time on all three treatment days, then regularly in clinical practice, in order to improve the tolerability of the dialysis, not all the overhydration is withdrawn in the treatment following the longer interval, rather only gradually in the both remaining dialysis sessions of the week. In these cases, it is advantageous, by storing mᵢₙₜₑᵣₙ and

$${V}_{FWR , d} = \frac{{V}_{FWR}}{{N}_{d}}$$

and by averaging the values over all values, e.g., of one week, to calculate the respective values, wherein N represents the number of dialysis treatments and N_{w} the number of days in the averaging period:

$${\overline{m}}_{inter , d} - \frac{1}{{N}_{W}} \frac{1}{N} {\sum }_{j = 1}^{N} {m}_{inter , i} , {\overline{V}}_{FWR , d} - \frac{1}{{N}_{W}} \frac{1}{N} {\sum }_{j = 1}^{N} {V}_{FWR , i} \qquad Formula 14$$

For example, with regard to the display of what has been calculated, the following thoughts may be used in the sense of the present disclosure:

Thus, to calculate and/or display the interdialytic, daily and weekly salt intake or fluid amount, several or some of the following and part of the parameters mentioned supra are considered:
V: distribution volume, i.e., the water content of the patient after removing the overhydration. This may be determined from anthropometric formulas (e.g., Watson-formula) or by bioimpedance measurements;
ΔV: interdialytic liquid excess corresponding to the interdialytic weight gain. Often, a liquid withdrawal V_{UE}=ΔV is prescribed for the dialysis;
N_{d}: number of days between the end of the previous dialysis and the current dialysis, determined by the current date and the stored date of the previous dialysis;
cₚᵣₑ(n): plasma sodium concentration at the beginning of the dialysis, determined from laboratory measurements or from conductivity-based online determination by OCM (cf. the EP 3 183 013 A1 or WO 2016/026569 A1 mentioned herein)
cₚₒₛₜ(n−1): plasma sodium concentration at the end of the previous dialysis, determined from laboratory measurements or from conductivity-based online determination by OCM. This value is stored at the end of the previous dialysis as patient-related value on a suitable storage medium and is made available with the current dialysis;
Vᵤᵣᵢₙₑ: accumulated urine residual excretion between the dialysis sessions. Determined by the patient at home. With constant habits, a representative value may be stored for the patient, advantageously as a daily amount so that the total amount may be calculated therefrom for differently long interdialytic intervals;
cᵤᵣᵢₙₑ: sodium concentration in the urine, determined from laboratory measurements or as an assumption from plausibility considerations. For example, this value and its possible error may be estimated depending on Vᵤᵣᵢₙₑ: since there is a severe renal insufficiency in the dialysis patient, cₚᵣₑ(n) may be assumed as upper limit. As lower limit, the dilution capacity of the kidney, possible in this state, may be assumed, e.g., cᵣₚₑₘᵢₙ=80 mmol/l;

All of the parameters or their values mentioned herein may either be entered directly at the dialysis machine or medical blood treatment apparatus or read by the dialysis machine from external sources (storage medium, network, etc.). It is likewise possible that the calculation of the interdialytic, daily and average daily salt intake takes place on external devices and that the measured values of the medical blood treatment apparatus, necessary for the calculation, are transmitted continuously to the external device.

One aspect of the representation on the display device of the medical blood treatment apparatus or on an external medium is that an estimated value of the parameter of interest is displayed as soon as possible. If during the dialysis session more data are available, e.g., from the determinations of the plasma sodium concentration or of the interdialytic salt transfer, this estimated value is refined such that by the end of the dialysis there is available the value with the highest possible accuracy. The current accuracy of the value may be indicated by numerically stating an error (e.g., standard deviation) or a possible range of values (e.g., confidence interval) or by a different indication of the reliability of the value, e.g., in color according to a traffic light model.

In several embodiments, initially, the calculation of the interdialytic and/or daily salt intake takes place separately for the part of the residual diuresis mᵤᵣᵢₙₑ. If both Vᵤᵣᵢₙₑ and cᵤᵣᵢₙₑ are known, then mᵤᵣᵢₙₑ=Vᵤᵣᵢₙₑcᵤᵣᵢₙₑ is directly calculated.

If only Vᵤᵣᵢₙₑ is known, the maximum salt excretion via the urine is calculated, for example, based on the plasma sodium:

$${m}_{urine , max} = {V}_{urine} {c}_{pre} ( n ) \qquad Formula 15$$

In this case, an estimated value for mᵤᵣᵢₙₑₘₐₓ may first be calculated by using for cₚᵣₑ(n) a value being typical for dialysis patients, e.g., 138 mmol/l or a value from the laboratory measurements or calculations from previous treatments.

Likewise, a lower limit mᵤᵣᵢₙₑₘᵢₙ is calculated. This may be a fixed proportion of cₚᵣₑ(n) or a function of the residual excretion Vᵤᵣᵢₙₑ and cₚᵣₑ(n), for example a linear function assuming a maximum dilution cᵤᵣᵢₙₑₘᵢₙ in a residual diuresis that exceeds a maximum value Vᵤᵣᵢₙₑₘₐₓ and assuming that with decreasing residual diuresis cᵤᵣᵢₙₑ is approaching to cₚᵣₑ(n).

$${m}_{urine , min} = \frac{{V}_{urine}}{{V}_{urine , max}} {c}_{urine , min} + ( 1 - \frac{{V}_{urine}}{{V}_{urine , max}} ) {c}_{pre} ( n ) \qquad Formula 16$$

For Vᵤᵣᵢₙₑ>Vᵤᵣᵢₙₑₘₐₓ there is for example assumed: mᵤᵣᵢₙₑₘᵢₙ=Vᵤᵣᵢₙₑcᵤᵣᵢₙₑₘᵢₙ.

The part of the interdialytic salt intake mᵢₙₜₑᵣ(Vᵤᵣᵢₙₑ=0) which has to be removed or is already removed by the dialysis, may also first be estimated in different ways and then be given in the course of the treatment with increasing accuracy:
mᵢₙₜₑᵣ(Vᵤᵣᵢₙₑ=0)=V_{UE}cₚᵣₑₛₑₜ wherein cₚᵣₑₛₑₜ is an estimated typical value of the predialytic plasma sodium, e.g., 138 mmol/l, or a value derived from past treatments;
Also known c_{LOSS}(n−1) estimation, e.g., by Formula 1 with estimated value for cₚᵣₑ;
If cₚᵣₑ has been determined in the course of the treatment from measurements based on conductivity, then this value may replace the previously used estimated value;
In the further course, the actual salt removal according to Formula 4 and Formula 5 or Formula 8 may be used for the calculation. This calculation can be used after reaching the prescribed ultrafiltration amount. In the meantime, the initially estimated value may be continuously corrected by the current measured value, wherein the measured value may be weighted with the ratio of the currently reached ultrafiltration amount to the prescribed ultrafiltration amount.

$${m}_{inter} ( {V}_{urine} = 0 ) = \frac{{V}_{UF} ( c )}{{V}_{UF , tot}} ( {m}_{UF} ( c ) - {V}_{UF} ( c ) \frac{{m}_{diff} ( c )}{V} ) + ( 1 - \frac{{V}_{UF} ( c )}{{V}_{UF , tot}} ) {{m}_{inter} ( {V}_{urine} = 0 )}_{est} \qquad Formula 17$$

In several embodiments, the calculating and displaying of the total amount of mᵢₙₜₑᵣ, with respect to the interdialytic, daily or average daily salt intake, may be done from the parts being calculated exemplarily in this way for the residual diuresis and the amount removed by the dialysis.

For displaying the interdialytic “free water removal” and the excess interdialytic drinking amount, the current “free water removal” V_{EWR} may be repeatedly or continuously calculated and displayed during the dialysis session, for example according to Formula 10, with multiple or continuous determination of m_{diff} (e.g., as described in EP 2 413 991 B1, the content of which is hereby also incorporated in its entirety by reference as the subject-matter of this disclosure).

For cₚᵣₑ, there may herein in turn be used, before a value which is determined during the treatment according to a known method (such a method is described in EP 3 183 013 A1 (published as WO 2016 026569 A1)), the content of which is also incorporated herein in its entirety by reference as the subject-matter of this disclosure) is provided, a population-related estimated value (e.g., 138 mmol/l), a patient-related historical value or a laboratory value for the time being.

Alternatively, V_{excessdrink}=V_{FWR} may be calculated also according to Formula 11, in that the value of the plasma sodium c_{LOSS}(n−1) which has been automatically determined at the end of the previous dialysis, is read from an internal or external storage medium.

Displaying V_{FWR} based on estimated values may in turn be optically marked. After providing an interdialytic measured value for cₚᵣₑ, then the latter is used for further calculation at least in the current dialysis session.

In support of nutritional counseling, with V_{excessdrink}=V_{FWR} may also be indicated already during the treatment but in any case, at the end of the treatment, if there has been a prescription of the salt transfer m_{diff} at the dialysis machine. This prescription may also be done by prescribing a relative change of the plasma sodium Δc according to m_{diff}=VΔc.

In addition to V_{FWR} also V_{FWRd} and V̅_{FWRd} may be displayed.

In this, displaying and calculating using the calculation device may be carried out both directly and as part of the medical blood treatment apparatus.

In several embodiments, the knowledge of the interdialytic salt intake mᵢₙₜₑᵣ and/or of the “free water removal” V_{FWR} may be used as dialysis prescription. A method suitable and optionally provided thereto is described below:

The values mᵢₙₜₑᵣ and V_{excessdrink} displayed, e.g., by the display device for informing those who are concerned, represent the salt intake and the water intake which have led to a (pathological) change of the plasma sodium.

In order to achieve a dialysis prescription which is easier to execute, it is now possible to use these values directly for therapy prescription. Such a prescription optionally replaces then the hitherto conventional prescription of ultrafiltration amounts and/or dialysis liquid sodium concentrations.

For example, the ultrafiltration amount V_{UE} may be calculated from mᵢₙₜₑᵣ(Vᵤᵣᵢₙₑ=0) according to Formula 6, as soon as cₚᵣₑ has been determined or estimated.

Similarly, the corresponding diffusive salt transfer m_{diff} may be calculated from the prescription of a “free water removal” V_{FWR} according to Formula 10. A control algorithm, e.g., as described in the above-mentioned EP 2 413 991 B1, may then adapt the dialysis liquid sodium during the treatment such that m_{diff} is achieved in the course of the treatment, e.g., at the end.

For purely exemplary application examples, reference is also made to the attached figures and/or their description.

The present disclosure further relates to a method for determining the interdialytic sodium intake of a patient and/or for determining the, for example not triggered or induced osmotically, interdialytic liquid intake, wherein the method encompasses: calculating the interdialytic sodium intake of said patient and/or calculating a, for example, not triggered osmotically, interdialytic liquid intake of said patient; and optionally sending a signal for controlling or closed-loop controlling of a communication device and/or a medical blood treatment apparatus.

In several embodiments, the method encompasses calculating the interdialytic sodium intake of the dialysis patient and/or his interdialytic liquid intake based on the plasma sodium concentration prevalent at the beginning of the dialysis session (n) and/or on the sodium concentration in the urine.

In several embodiments, the method encompasses querying or requesting several times (the values measured by the medical blood treatment apparatus during a dialysis session (n) carried out by the blood treatment apparatus), e.g., using the medical blood treatment apparatus, in order to, based thereon, repeatedly or more precisely calculate the interdialytic sodium intake and/or the interdialytic liquid intake.

In several embodiments, the method encompasses stating a prescription based on the interdialytic sodium intake and/or the interdialytic liquid intake.

In some embodiments, the method encompasses one or several of the method steps mentioned herein or steps executed in arbitrary combination by one of the apparatuses mentioned herein, for example when used as intended or according to their suitability or configuration.

One or several of the advantages mentioned herein may be achievable by some embodiments, including the following:

As stated at the beginning and proven by numerous studies, increased table salt intake may lead to an increased occurrence of cardiovascular diseases and to cardiovascular incidents such as heart attack and stroke.

The table salt taken with the nutrition, is mostly excreted in the common population via the urine, wherein the kidney has the possibility to regulate the salt content in the urine such that both an excess and a relative lack of sodium in the blood may be compensated.

These harmful mechanisms associated with salt intake are also relevant for patients with absent or severely impaired renal function. Since in the case of said patients a salt excretion with the urine is only limited or no longer possible, an increased salt intake leads additionally to an increased drinking amount due to the sensation of thirst resulting therethrough which leads to an overhydration of the patient. This leads to a burden or strain on the circulatory system and manifests itself, e.g., in edema and water retention in the lung. This overhydration is reduced again, in patients with renal insufficiency, in the course of the hemodialysis, which is typically carried out several times per week, through removal of liquid by ultrafiltration. The higher the amount of liquid to be withdrawn, the higher the withdrawal rate must be because of the usually fixed dialysis duration.

However, with higher withdrawal rates, the risk of interdialytic (i.e., during a dialysis or dialysis session) blood pressure drops also increases leading also to long-term damage.

Table salt is contained in many foods, for example also hidden in convenience products, where it serves as a cheap seasoning. Thus, the salt content of some ready-made pizzas already corresponds to the WHO recommended daily salt intake of ca. 5 g NaCl. Despite the information on many foods to their salt content, these are often ignored and only the personal adding of salt is perceived as salt intake. Thus, many dialysis patients, despite regularly prescribed high ultrafiltration levels, are unaware that the cause for this is ultimately the (hidden) intake of salt during the interdialytic interval.

Therefore, it is the task of the treating dialysis doctor and his nursing staff to work in these cases towards reducing the amount of salt intake by a nutritional counseling. As discussed, the amount of salt intake is often not fully consciously perceived, making it difficult to argue with the patient.

It is essential for the success of nutritional counseling to give the best estimate possible of the interdialytic and daily salt intake. The methods and systems disclosed herein may be of valuable assistance here. In addition, it is particularly suitable for use by the patient himself to obtain information about his food intake and in particular his salt or water intake, should professional advice not be available. The methods and systems disclosed herein can also give the patient the best estimate possible of the physiologically unnecessary and potentially harmful drinking amount.

In addition to the drinking triggered by the sensation of thirst caused by salt intake, referred to herein as osmotically controlled drinking, some patients also drink for other reasons (habit, “social drinking”, etc.). In addition to an increase in overhydration, this leads to a decrease in plasma sodium concentration, which represents a pathological condition. The methods and systems disclosed herein may also advantageously contribute to recognizing that quantities of liquid are consumed that are beyond the quantities triggered osmotically due to the salt intake.

In this, the methods and systems disclosed herein may advantageously differentiate themselves from, e.g., the idea of a manual calculation based on the assumption that the prescribed liquid withdrawal corresponds to the interdialytic weight gain and that no interdialytic salt has been excreted in any other way. If, in such a procedure, e.g., the liquid withdrawal carried out during an ultrafiltration treatment is multiplied by a typical sodium concentration in blood, e.g., 138 mmol/l, in order to estimate the removed sodium amount, then this would not take into account neither the patient's individual deviation of the plasma sodium concentration nor any possible excretion via a residual renal function. In this regard, the methods and systems described herein may provide advantages as described above.

Thus, by the present disclosure, the interdialytic and daily (dietary) sodium intake as well as the non-osmotically triggered drinking amount may advantageously be calculated and displayed, even in the case of patients with residual diuresis. For example, the following may be possible:

The calculation and representation of the interdialytic salt and/or liquid intake, wherein the latter is related to deviations from the “ideal” drinking amount, i.e., the drinking amount which does not change the plasma sodium concentration;

The consideration of the residual diuresis, determined from external data, in the calculation;

The reference or relation of these quantities to daily or average daily quantities, thus, storing of and accessing values of previous treatments;

Displaying as early as possible these values during the dialysis, by first using estimates which are then gradually replaced with increasing information input by calculations based on measured values;

The possibility to calculate and display on both the medical blood treatment apparatus and an external computing and display unit which may also be used ambulatory.

### BRIEF DESCRIPTION OF THE DRAWINGS

In the following, the present disclosure is described based on embodiments thereof with reference to the accompanying drawings. The following applies in the figures:

FIG. 1 shows, in a schematically simplified manner, sections of a medical blood treatment apparatus in an exemplary embodiment, exemplarily connected to a blood cassette for executing a patient's treatment, controlled and/or closed-loop controlled by a control device which is connected in signal communication to a calculation device;

FIG. 2 shows a user interface as part of a calculation device; and

FIG. 3 shows an exemplary calculation device.

### DETAILED DESCRIPTION

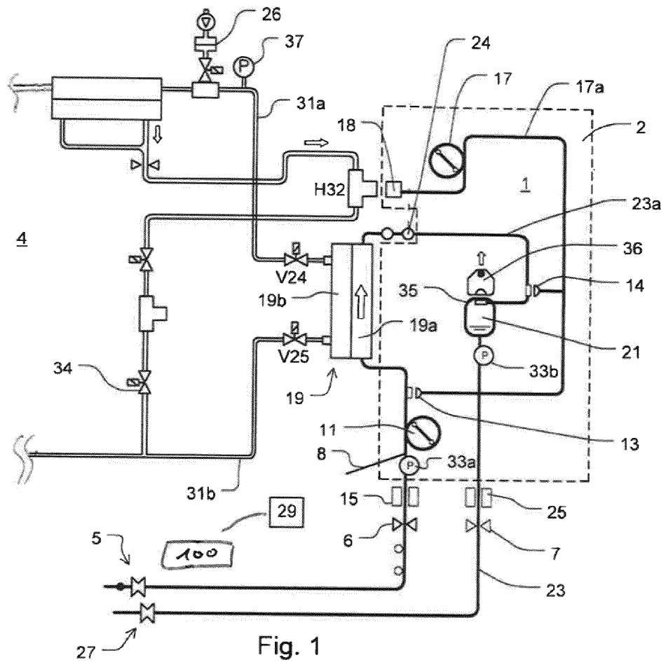

FIG. 1 shows an extracorporeal blood circuit 1, which would be connected for a treatment to the vascular system of the patient (not shown) via double-needle access or via single-needle access using, e.g., an additional Y-connector. The blood circuit 1 is provided optionally in sections thereof in or on a blood cassette 2. This blood cassette 2 is designed to be used also in other treatment types, e.g., a single-needle treatment.

Pumps, actuators, and/or valves in the area of the blood circuit 1 are connected to a blood treatment apparatus 4 or to a control device 29 included by it.

The blood circuit 1 includes an arterial patient hose clamp 6 and an arterial connection needle 5 (as an example for an access device) of an arterial section or of an arterial patient line or blood withdrawal line 9. The blood circuit 1 further includes a venous patient hose clamp 7 and a venous connection needle 27 (as an example for a further or second access device) of a venous section or of a venous patient line or blood return line 23.

A blood pump 11 is provided in the arterial section 9 and a substituate pump 17 is connected to a substituate line 17a. The substituate line 17a can be connected with a substituate source through an, e.g., automatic, substituate port 18. By the substituate pump 17, substituate may be introduced via pre-dilution or via post-dilution through associated lines 13 or 14 into line sections, e.g., into the arterial section 9 or into a venous section 23a (between a blood chamber 19a of a blood filter 19 and a venous air separation chamber 21), of the blood circuit 1.

The blood filter 19 includes the blood chamber 19a which is connected to the arterial section 9 and to the venous section 23. A dialysis liquid chamber 19b of the blood filter 19 is connected to a dialysis liquid inlet line 31a leading to the dialysis liquid chamber 19b and to a dialysate outlet line 31b leading away from the dialysis liquid chamber 19b.

The dialysis liquid inlet line 31a optionally includes a valve V24 by which the flow within the dialysis liquid inlet line 31a may be interrupted. The dialysate outlet line 31b optionally includes a valve V25 by which the flow within the dialysate outlet line 31b may be interrupted.

The dialysis liquid inlet line 31a is further optionally connected to a compressed air source 26 by another internal valve of the apparatus. The compressed air source 26 may be provided as a component of the blood treatment apparatus 4 or as a part separated thereof. A pressure sensor 37 may be provided downstream of the compressed air source 26.

A venous connection with, e.g., the venous section 23 or 23a may be achieved by a factory-provided optional connection section 24.

The control device 29 may be part of or embody a control device of the blood treatment apparatus 4.

The arrangement of FIG. 1 includes an optional detector 15 for detecting air and/or blood. The arrangement of FIG. 1 further includes one or two pressure sensors 33a, 33b at the illustrated points in FIG. 1. Further pressure sensors may be provided, e.g., the pressure sensor 37.

In FIG. 1, the single-needle chamber 36 is used as buffer and/or compensation tank during or after a single-needle method during which the patient is connected to the extracorporeal blood circuit 1 via only one of the two blood lines 9, 23.

The arrangement in FIG. 1 additionally includes an optional detector 25 for detecting substituate and/or blood.

In FIG. 1, the calculation device 100 is exemplarily shown in signal communication with the control device and/or closed-loop control device 29.

The present invention is not limited to the embodiments as described herein, this is considered only for illustrative purposes.

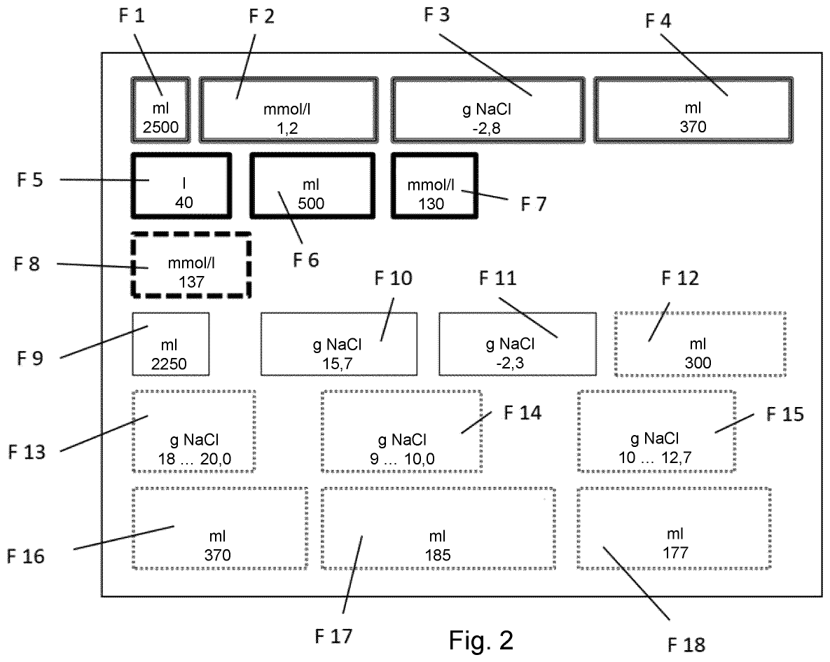

FIG. 2 shows an exemplary user interface as an example for an input device 103 and/or a display device 109 of a calculating device 100 for displaying and prescribing the salt and liquid balance.

Such a user interface may be positioned directly on the hemodialysis apparatus and/or on an external display and computing unit (computer, laptop, tablet device, etc.) connected to the dialysis apparatus. In this example, the display and input elements are arranged in series by function, although also other arrangements, labelings, etc. are possible. Also, not all elements must be present, or also further data may be represented respectively. These may either be additional dialysis parameters or the conversion of values of the represented parameters in other units.

Line 1 encompasses input elements for prescribing a hemodialysis treatment (includes HD and all types of HDF and HF):
V(UF) in field F1 of FIG. 2: prescription of liquid removal by ultrafiltration. The volume set by the doctor will be withdrawn from the patient until the end of the treatment.
Target plasma sodium change (field F2), target diffusive Na removal (field F3), target free water removal (field F4): These three input elements may be used alternatively, since the quantities contained herein are in a fixed relationship to each other: As described in the formulas, they can be converted into each other with the additional knowledge of V(urea) and plasma sodium (pre). It is also possible to select the displayed physical unit within the elements using internally stored conversion factors (e.g., conversion “mmol NaCl” into “g NaCl”). Negative values at the diffusive Na removal mean a sodium transfer into the patient, corresponding to a “free water removal”.

Line 2 encompasses input elements for the input of auxiliary quantities which are required to convert the prescription from line 1 into specific settings of dialysis parameters, for example the dialysis liquid sodium concentration, or to be able to determine the physiologically relevant quantities of lines 4 to 6 from measured quantities of the dialysis apparatus:
V(urea) (field F5): distribution volume of the patient. For converting concentration changes in the patient to substance amounts.
Daily residual diuresis (field F6), Na in the urine (field F7): For calculating the total salt intake.

Line 3 encompasses input elements, whose values may come from different sources:
Plasma sodium (pre) (field F8): Patient's plasma sodium at the beginning of the dialysis. This value may be preallocated from electronic records, e.g., from current or historical laboratory data. Also, a manual input at the beginning of the treatment is possible. In the course of the treatment, this value is automatically determined by the apparatus and the initial value is replaced by the determined value.

Line 4 encompasses display elements, which characterize the progress of the treatment:
V(UF) act (field F9): amount of liquid withdrawn by ultrafiltration up to the present point of time
total Na removal (field F10): total amount of salt withdrawn by ultrafiltration and diffusion up to the present point of time (possible display in different units)
Na removal diffusive (field F11): amount of salt withdrawn by diffusion up to the present point of time. This may be positive or negative. A negative value corresponds to salt transfer into the patient.
Free water removal (field F12): Reached volume of “free water removal” up to the present point of time. Since the free water removal is included in the here displayed value plasma sodium (pre), which is initially only present as an estimated value, there may be carried out in addition to the numerical display of the value a marking whether this is a preliminary calculation based on estimated values or an updated or even final calculation based on intradialytic measurements.

Line 5 encompasses display elements for salt intake of the patient:
“Interdialytic salt intake” (field F13): calculated from the residual diuresis and sodium in the urine, as well as the total sodium removal (ultrafiltration and diffusion) during dialysis. At the beginning of the treatment, the prescription values are used hereby and the displayed result, as in the indication of “free water removal”, is marked as an estimated value. Until the end of the treatment, the accuracy will then be gradually improved with measured values of the sodium balance obtained during the treatment, which may also be visually identified. In addition, because of the uncertainty of the sodium content in the urine, a range of values corresponding to a confidence interval may be displayed instead of a single value for the interdialytic salt intake.
“Daily salt intake since the last dialysis” (field F14): As described, display modalities as supra. The number of days required for the calculation since the previous dialysis may be determined by accessing an internal or external storage medium, which receives the date of the previous dialyses of the patient, or may be entered using the input device 103.
“Average daily salt intake” (field F15): as described, display modalities as supra. For this purpose, the values determined in the previous dialysis are used, e.g., by accessing an external or internal storage medium or after an input.

Line 6 encompasses display elements for the drinking amount of the patient:
“Interdialytic excess drinking amount” (field F16): Drinking amount which has led to a change in plasma sodium concentration; positive when the plasma sodium concentration has decreased. The calculation may be carried out in accordance with Formula 10, based on the current prescription, or in accordance with Formula 11 based on the plasma sodium value at the end of the previous treatment read from an internal or external storage medium. As with the values in line 5, the temporary use of estimated values may be optically represented.
“Daily excess drinking amount since the last dialysis” (field F17): Calculation analogously to “daily salt intake since the last dialysis”
“Average daily excess drinking amount” (filed F18): Calculation analogously to “average daily salt intake” from field F15

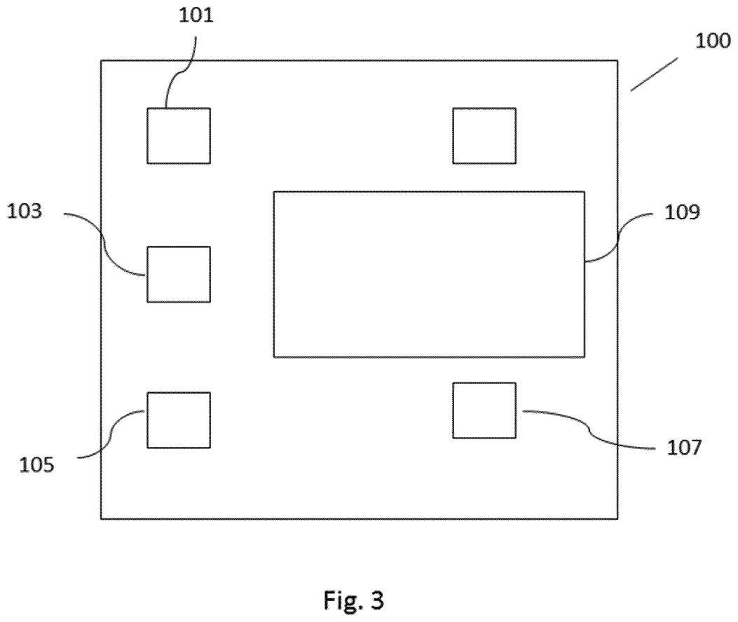

FIG. 3 shows a calculation device 100 for determining an interdialytic sodium intake mᵢₙₜₑᵣ of a patient and/or for determining a mostly non-osmotically triggered interdialytic liquid intake V_{excess drink}.

The calculation device 100 includes a storage device 101 and/or an input device 103. They serve for storing or entering parameter values of the patient.

The calculation device 100 further includes a computing device 105. It is configured for calculating the interdialytic sodium intake mᵢₙₜₑᵣ of the patient and/or for calculating his non-osmotically triggered interdialytic liquid intake V_{excess drink}. Corresponding algorithms and formulas, such as disclosed herein, may for this purpose be stored in the computing device 105 or read by it from suitable sources such as the storage device 101.

Finally, the calculation device 100 includes an output device 107. It serves to output a signal for controlling or closed-loop controlling an optional communication device 109 and/or the medical blood treatment apparatus 4.

The communication device 109 may be configured for wired or wireless signal connection with a component of the medical blood treatment apparatus 4, e.g., its control or closed-loop control device 29.

The communication device 109 may additionally or alternatively be connected to an optionally provided display device or may be designed as such. The display device 109 may be, or may include, the user interface shown in FIG. 2.

The communication device 109 may, for example, be designed as a display device, be part of the calculation device 100 or of the medical blood treatment apparatus 4.

Where it is not technically impossible, several of the aforementioned apparatuses may be combined into a single unit.

### LIST OF REFERENCE NUMERALS

1 extracorporeal blood circuit
2 blood cassette
4 blood treatment apparatus
5 access device, for example arterial connection needle
6 arterial patient hose clamp
7 venous patient hose clamp
8 inlet line
9 arterial section or arterial blood withdrawal line or arterial patient line
11 blood pump
13 addition site for substituate (pre-dilution)
14 addition site for substituate (post-dilution)
15 arterial air-blood-detector
17 conveying device, for example a substituate pump
17a substituate line
18 automatic substituate port
19 blood filter
19a blood chamber
19b dialysis liquid chamber
21 venous air separation chamber
23 venous section or venous blood return line or venous patient line
23a venous section
24 connecting site, connecting section
25 venous substituate-blood-detector
26 compressed air source
27 access device, for example venous connection needle
29 control device and/or closed-loop control device
31a dialysis liquid inlet line
31b dialysate outlet line
33a pressure sensor
33b pressure sensor
35 single needle valve
36 single needle chamber
37 pressure sensor
41 connector
100 calculation device
101 storage device
103 input device
105 computing device
107 output device
109 communication device and/or display device
F 1 bis F 16 display fields of the interface
V24 valve
V25 valve

1-11. (canceled)

12. A calculation device for determining an interdialytic sodium intake of a patient and/or for determining an interdialytic liquid intake, the calculation device comprising:
a storage device and/or an input device configured for storing or entering parameter values of said patient;
a computing device, configured for calculating the interdialytic sodium intake of the patient and/or for calculating an interdialytic liquid intake of said patient; and
an output device for outputting a signal to control or closed-loop control a communication device and/or a medical blood treatment apparatus.

13. The calculation device according to claim 12, wherein the computing device is configured for calculating the interdialytic sodium intake and/or the interdialytic liquid intake based on a plasma sodium concentration prevalent at a beginning of a dialysis session.

14. The calculation device according to claim 12, wherein the computing device is configured for calculating the interdialytic sodium intake and/or the interdialytic liquid intake based on a sodium concentration in urine.

15. The calculation device according to claim 12, wherein the computing device is configured to be in signal communication with the medical blood treatment apparatus such as to repeatedly query or receive from the medical blood treatment apparatus, during a dialysis session carried out by the medical blood treatment apparatus, values measured by the medical blood treatment apparatus, in order to, based thereon, repeat or specify the calculation of the interdialytic sodium intake and/or of the interdialytic liquid intake.

16. The calculation device according to claim 12, wherein the output device is configured to display, output, and/or store the interdialytic sodium intake and/or the interdialytic liquid intake on a display device.

17. The calculation device according to claim 16, wherein the display device is a communication device.

18. The calculation device according to claim 17, wherein the communication device comprises at least one of: a monitor, a display, a printer, a storage element, or a database.

19. The calculation device according to claim 17, wherein the output device is configured to output, store, and/or to display on the display device, calculated values, wherein the calculated values comprise the interdialytic sodium intake and/or the interdialytic liquid intake, while also stating a qualitative and/or quantitative accuracy of the displayed, outputted, and/or stored value.

20. The calculation device according to claim 19, wherein stating the qualitative and/or quantitative accuracy of the displayed value is or includes specifying a value range, a confidence interval, a representation in one of a plurality of predetermined colors, the size of an error, an uncertainty, a possible value range, a confidence interval, a standard deviation, a variance, a color coding, and/or a traffic light display.

21. The calculation device according to claim 20, wherein the color coding comprises multiple colors.

22. The calculation device according to claim 12, wherein the output device for outputting a signal for controlling of a communication device is configured for stating a prescription based on the interdialytic sodium intake and/or on the interdialytic liquid intake.

23. The calculation device according to claim 12, wherein the output device is configured for controlling the medical blood treatment apparatus, via the signals outputted by said output device to the medical blood treatment apparatus, such that a dialysis session ends when or once a determined interdialytic sodium intake and/or liquid intake has been withdrawn from treated blood.

24. The calculation device according to claim 12, configured for reading values of at least one or several of the following parameters: distribution volume of the patient; interdialytic liquid excess corresponding to interdialytic weight gain; plasma sodium concentration at the beginning of a dialysis session; plasma sodium concentration at an end of a previous dialysis session; accumulated residual urine excretion between dialysis sessions; sodium concentration in urine; and number of days between the end of the previous dialysis session and the current dialysis session, determined by a current date and a stored date of a previous dialysis session.

25. A medical blood treatment apparatus comprising:
fluid lines which encompass at least one dialysis liquid inlet line and/or a dialysate outlet line;
at least one conveying device for conveying a dialysis liquid within the dialysis liquid inlet line and/or within the dialysate outlet line; and
at least one control device and/or closed-loop control device,
wherein the medical blood treatment apparatus is configured to be connected to a dialysis liquid chamber of a blood filter respectively by the dialysis liquid inlet line and the dialysate outlet line, wherein the blood filter comprises a blood chamber, wherein dialysis liquid chamber and blood chamber are separated from each other by a semipermeable membrane, and
wherein the control device and/or closed-loop control device is configured for prompting or effecting a blood treatment using the medical blood treatment apparatus by hemofiltration, hemodialysis, or hemodiafiltration, for the purpose of which the control device and/or the closed-loop control device is connected to in signal transmission or comprises a calculation device comprising:
a storage device and/or an input device configured for storing or entering parameter values of said patient;
a computing device, configured for calculating the interdialytic sodium intake of the patient and/or for calculating an interdialytic liquid intake of said patient; and
an output device for outputting a signal to the control device and/or closed-loop control device.

26. The medical blood treatment apparatus according to claim 25, suitable and/or configured to execute a hemodialysis, a hemofiltration, a hemodiafiltration, or a separation method.

27. The medical blood treatment apparatus according to claim 25, wherein the computing device is configured for calculating the interdialytic sodium intake and/or the interdialytic liquid intake based on a plasma sodium concentration prevalent at a beginning of a dialysis session.

28. The medical blood treatment apparatus according to claim 25, wherein the computing device is configured for calculating the interdialytic sodium intake and/or the interdialytic liquid intake based on a sodium concentration in urine.

29. The medical blood treatment apparatus according to claim 25, wherein the computing device is configured to be in signal communication with the medical blood treatment apparatus such as to repeatedly query or receive from the medical blood treatment apparatus, during a dialysis session carried out by the medical blood treatment apparatus, values measured by the medical blood treatment apparatus, in order to, based thereon, repeat or specify the calculation of the interdialytic sodium intake and/or of the interdialytic liquid intake.

30. The medical blood treatment apparatus according to claim 25, wherein the output device is configured to display, output, and/or store the interdialytic sodium intake and/or the interdialytic liquid intake on a display device.

31. The medical blood treatment apparatus according to claim 25, wherein the calculating device is configured for reading values of at least one or several of the following parameters: distribution volume of the patient; interdialytic liquid excess corresponding to interdialytic weight gain; plasma sodium concentration at the beginning of a dialysis session; plasma sodium concentration at an end of a previous dialysis session; accumulated residual urine excretion between dialysis sessions; sodium concentration in urine; and number of days between the end of the previous dialysis session and the current dialysis session, determined by a current date and a stored date of a previous dialysis session.

32. The medical blood treatment apparatus according to claim 25, wherein the at least one dialysis liquid inlet line and the dialysate outlet line are connected in fluid communication with each other by a connector.
